(19) United States
(12) Patent Application Publication
(10) Pub. No.: US 2019/0309258 A1
(43) Pub. Date: Oct. 10, 2019

(54) ADVANCED THIN PROTECTIVE FILMS

(71) Applicant: Norma Arcelia Alcantar, Tampa, FL (US)

(72) Inventor: Norma Arcelia Alcantar, Tampa, FL (US)

(73) Assignee: University of South Florida, Tampa, FL (US)

(21) Appl. No.: 16/376,525

(22) Filed: Apr. 5, 2019

Related U.S. Application Data
(60) Provisional application No. 62/653,100, filed on Apr. 5, 2018.

(51) Int. Cl. C12N 5/00

(52) U.S. Cl. CPC C12N 5/0068; C12N 2537/10; C12N 2539/10; C12N 2533/70

(57) ABSTRACT

Thin film coatings and methods of manufacture thereof are presented. Ultra-thin coatings of cross-linked high-methoxyl pectin polysaccharides were fabricated by spin-casting solutions of citrus pectin followed by cross-linking upon exposure to solutions of calcium chloride (CaCl₂) in ethanol. By adjusting temperature, degree of cross-linking, and pH of the surroundings, the pectin coatings can be carefully tuned for a desired response.

### CROSS REFERENCE TO RELATED APPLICATIONS

This application is a nonprovisional of and claims priority to U.S. Provisional Patent Application Ser. No. 62/653,100, entitled “Advanced Cutin Thin Protective Films (ACT-PT)”, filed Apr. 5, 2018, the contents of which are hereby incorporated by reference into this disclosure.

### GOVERNMENT SUPPORT

This invention was made with Government support under Grant Nos. 1512225 and 1560579 awarded by the National Science Foundation (NSF). The Government has certain rights in the invention.

### FIELD OF INVENTION

This invention relates to protective films. Specifically, the invention provides a thin protective film made from cacti cutin or pectin polysaccharides.

### BACKGROUND OF THE INVENTION

Thin films or coatings of biologically derived pectin polysaccharides could have potential in many applications such as controlled cell attachment, controlled release of drugs, tissue engineering scaffolds, and membranes for mass-separation of organic compounds, etc. (Toomey, R.; Freidank, D.; Rae, J., Swelling behavior of thin, surface-attached polymer networks. Macromolecules 2004, 37 (3), 882-887; Tokarev, I.; Minko, S., Stimuli-responsive hydrogel thin films. Soft Matter 2009, 5 (3), 511-524; Zhang, Q.; Archer, L. A., Interfacial friction and adhesion of cross-linked polymer thin films swollen with linear chains. Langmuir 2007, 23 (14), 7562-7570; Harmon, M. E.; Kuckling, D.; Frank, C. W., Photo-cross-linkable PNIPAAm copolymers. 2. Effects of constraint on temperature and pH-responsive hydrogel layers. Macromolecules 2003, 36 (1), 162-172). Pectin is a naturally occurring water-soluble heteropolysaccharide extracted from cell walls and intercellular regions of plants functioning as the hydrating component of the cellulosic network. (Voragen, A. G.; Coenen, G.-J.; Verhoef, R. P.; Schols, H. A., Pectin, a versatile polysaccharide present in plant cell walls. Structural Chemistry 2009, 20 (2), 263; Goycoolea, F. M.; Cardenas, A., Pectins from Opuntia spp.: a short review. Journal of the Professional Association for Cactus Development 2003, 5 (1), 17-29). Pectin polysaccharides can be extracted from several natural sources such as cactus mucilage or orange peels ⁷⁻⁹, and are intrinsically biocompatible. (Maran, J. P.; Sivakumar, V.; Thirugnanasambandham, K.; Sridhar, R., Optimization of microwave assisted extraction of pectin from orange peel. Carbohydrate polymers 2013, 97 (2), 703-709; Thakur, B. R.; Singh, R. K.; Handa, A. K.; Rao, M., Chemistry and uses of pectin—a review. Critical Reviews in Food Science & Nutrition 1997, 37 (1), 47-73; Burton, R. A.; Gidley, M. J.; Fincher, G. B., Heterogeneity in the chemistry, structure and function of plant cell walls. Nature Chemical Biology 2010, 6 (10), 724-732).

In general, the structure is mostly composed of D-galacturonic acid residues linked by α-1,4-glycosidic bonds, which are often interrupted with different sugars such as rhamnose, arabinans, galactose and xylose. (Round, A. N.; Rigby, N. M.; MacDougall, A. J.; Morris, V. J., A new view of pectin structure revealed by acid hydrolysis and atomic force microscopy. Carbohydrate Research 2010, 345 (4), 487-497; Maxwell, E. G.; Belshaw, N. J.; Waldron, K. W.; Morris, V. J., Pectin—an emerging new bioactive food polysaccharide. Trends in Food Science & Technology 2012, 24 (2), 64-73). The carboxyl group associated with the D-galacturonic acid residue exists in two primary forms: the carboxylate salt and the neutral methoxylated or ester form that has been neutralized with sodium, potassium or ammonium ions. (Ridley, B. L.; O'Neill, M. A.; Mohnen, D., Pectins: structure, biosynthesis, and oligogalacturonide-related signaling. Phytochemistry 2001, 57 (6), 929-967; Willats, W. G.; Knox, J. P.; Mikkelsen, J. D., Pectin: new insights into an old polymer are starting to gel. Trends in Food Science & Technology 2006, 17 (3), 97-104; Yapo, B. M., Pectic substances: From simple pectic polysaccharides to complex pectins—A new hypothetical model. Carbohydrate Polymers 2011, 86 (2), 373-385; Mohnen, D., Pectin structure and biosynthesis. Current opinion in plant biology 2008, 11 (3), 266-277). Depending on the degree of esterification, pectins are broadly classified as low methoxyl (<50% esterification) and high-methoxyl (≥50% esterification).

Pectin polysaccharides have been widely used in pharmaceutical formulations and in the food and beverage industries due to their non-toxic nature, ability to form gels, and low production costs. They can be readily cross-linked to obtain networks exhibiting sensitive conformations to internal and external variables. Gelling properties of bulk pectin networks are strongly governed by the methoxyl content [13,16-19]. (Willats, W. G.; Knox, J. P.; Mikkelsen, J. D., Pectin: new insights into an old polymer are starting to gel. Trends in Food Science & Technology 2006, 17 (3), 97-104; Kaya, M.; Sousa, A. G.; Crépeau, M.-J.; Sorensen, S. O.; Ralet, M.-C., Characterization of citrus pectin samples extracted under different conditions: influence of acid type and pH of extraction. Annals of botany 2014, mcu150; Morris, E.; Powell, D.; Gidley, M.; Rees, D., Conformations and interactions of pectins: I. Polymorphism between gel and solid states of calcium polygalacturonate. Journal of molecular biology 1982, 155 (4), 507-516; Jarvis, M. C., Structure and properties of pectin gels in plant cell walls. Plant, Cell & Environment 1984, 7 (3), 153-164; Munarin, F.; Tanzi, M.; Petrini, P., Advances in biomedical applications of pectin gels. International journal of biological macromolecules 2012, 51 (4), 681-689). Low-methoxyl pectins readily cross-link in the presence of divalent ions such as calcium with few constraints on the gelling conditions. (Liu, L.; Fishman, M. L.; Hicks, K. B., Pectin in controlled drug delivery—a review. Cellulose 2007, 14 (1), 15-24; Rinaudo, M., Physicochemical properties of pectins in solution and gel states. Progress in biotechnology 1996, 14, 21-33; Sriamornsak, P., Chemistry of pectin and its pharmaceutical uses: A review,” Silpakorn University International Journal 3.1-2 (2003): 206-228). The underlying mechanism is understood by the well-known ‘egg-box’ model, which describes the formation of networks through associations between Ca²⁺ and non-esterified regions of the pectin. (Powell, D.; Morris, E.; Gidley, M.; Rees, D., Conformations and interactions of pectins: II. Influence of residue sequence on chain association in calcium pectate gels. Journal of molecular biology 1982, 155 (4), 517-531; Walkinshaw, M.; Arnott, S., Conformations and interactions of pectins: II. Models for junction zones in pectinic acid and calcium pectate gels. Journal of Molecular Biology 1981, 153 (4), 1075-1085; Axelos, M.; Thibault, J., The chemistry of low-methoxyl pectin gelation. The chemistry and technology of pectin 1991, 109-118; Grant, G. T.; Morris, E. R.; Rees, D. A.; Smith, P. J.; Thom, D., Biological interactions between polysaccharides and divalent cations: the egg-box model. FEBS letters 1973, 32 (1), 195-198). The ionic bridges between Ca²⁺ and pectin carboxyl groups induce chain-chain associations to develop junction zones. As the degree of esterification is increased, calcium mediated cross-linking is no longer guaranteed and depends on several factors as the affinity for Ca²⁺ ions decreases. For instance, it was reported that Ca²⁺ gelation may be achieved in high-methoxyl pectins (68% esterification) if the non-esterified residues are sufficiently contiguous (or blocky) and the pectin concentration is high enough to induce chain entanglement. (MacDougall, A. J.; Needs, P. W.; Rigby, N. M.; Ring, S. G., Calcium gelation of pectic polysaccharides isolated from unripe tomato fruit. Carbohydrate Research 1996, 293 (2), 235-249). At these higher degrees of esterification, however, cross-linking is more readily achieved through non-ionic mechanisms, for example, by mixing the pectin with dissolved sugar at low pH. (Tibbits, C. W.; MacDougall, A. J.; Ring, S. G., Calcium binding and swelling behaviour of a high methoxyl pectin gel. Carbohydrate Research 1998, 310 (1), 101-107).

Despite the prevalence of bulk gels of pectin, coatings or thin films of cross-linked pectin have not been reported. (Li, Y.; Tanaka, T., Kinetics of swelling and shrinking of gels. The Journal of chemical physics 1990, 92 (2), 1365-1371; Hirose, H.; Shibayama, M., Kinetics of volume phase transition in poly (N-isopropylacrylamide-co-acrylic acid) gels. Macromolecules 1998, 31 (16), 5336-5342; Tanaka, T.; Fillmore, D. J., Kinetics of swelling of gels. The Journal of Chemical Physics 1979, 70 (3), 1214-1218). Accordingly, what is needed are thin films or coatings of cross-linked pectin as well methods of producing such coatings and thin films.

### SUMMARY OF INVENTION

Pectin polysaccharides have significant potential as all-natural “green” coatings that exhibit tunable responses. To this end, ultra-thin coatings of cross-linked high-methoxyl pectin polysaccharides were fabricated by spin-casting solutions of citrus pectin followed by cross-linking upon exposure to solutions of calcium chloride (CaCl₂) in ethanol. Ethanol is a poor solvent for citrus pectin, which does not disturb or disrupt the thin film coating yet allows for controlled diffusion of Ca²⁺ ions into the coatings to tune the extent of cross-linking. The pectin coatings were cross-linked over a range of CaCl₂ concentrations. The swelling of the cross-linked coatings in water was characterized by ellipsometry, which demonstrated that the equilibrium water content was dependent on the degree of cross-linking. Interestingly, it was found that the swelling of the high-methoxyl coatings was a strong function of temperature. At temperatures below approximately 35° C., the coatings were hydrophilic and absorbed water. At higher temperatures, however, the coatings expelled water and collapsed giving rise to distinctive de-swelling profiles. As temperature was increased, the coatings underwent a volume-phase transition, where water was rejected from coatings at higher temperatures, similar to a hydrophilic/hydrophobic transition found in lower critical solution temperature polymers. ATR-FTIR was used in parallel with ellipsometry to provide insights into the nature of the transition. The hydrophilic/hydrophobic transition was driven by dehydration of carbomethoxy groups along the backbone of the pectin chains, whereas water remained bound to the carboxylate groups. By adjusting temperature, degree of cross-linking, and pH of the surroundings, it was ultimately shown that the pectin coatings can be carefully tuned for a desired response.

The inventors have also developed advanced cutin thin protective films from cacti which are sustainable thin films that can be synthesized for functional coating applications with properties similar to the outer surface of cacti.

In an embodiment, a method of manufacturing a tunable crosslinked thin film coating is presented comprising: obtaining a polysaccharide having a high methoxyl content; spin-coating the polysaccharide onto a substrate to form a thin film; and exposing the thin film polysaccharide to a concentration of calcium chloride (CaCl₂) in ethanol to produce a crosslinked thin film coating.

The polysaccharide may be a heteropolysaccharide such as pectin. The pectin may have a degree of esterification of between about 67-70%.

The method may further be comprised of tuning the thin film coating according to the specific preparation needed. This tuning may be achieved in various ways. The concentration of CaCl₂ may be adjusted to tune an amount of crosslinking in the crosslinked thin film coating. The temperature the crosslinked thin film coating is exposed to may be adjusted in order to tune thickness of the crosslinked thin film coating. The pH of a solution the crosslinked thin film coating is exposed to may be adjusted in order to tune thickness of the crosslinked thin film coating.

In an embodiment, a crosslinked thin film coating is presented comprising: a polysaccharide crosslinked with calcium chloride (CaCl₂) wherein the crosslinked thin film coating is produced by the process comprising: obtaining a polysaccharide having a high methoxyl content; spin-coating the polysaccharide onto a substrate to form a thin film; and exposing the thin film polysaccharide to a concentration of CaCl₂ in ethanol to produce a crosslinked thin film coating.

The polysaccharide may be a heteropolysaccharide such as pectin. The pectin may have a degree of esterification of between about 67-70%.

Tuning the thin film coating according to the specific preparation needed may be achieved in various ways. The concentration of CaCl₂ may be adjusted to tune an amount of crosslinking in the crosslinked thin film coating. The temperature the crosslinked thin film coating is exposed to may be adjusted in order to tune thickness of the crosslinked thin film coating. The pH of a solution the crosslinked thin film coating is exposed to may be adjusted in order to tune thickness of the crosslinked thin film coating.

In a further embodiment, a thermo-responsive crosslinked coating for a substrate capable of use in cell culture and release applications comprising: a polysaccharide crosslinked with calcium chloride (CaCl₂) coating wherein the crosslinked coating is produced by the process comprising: obtaining a polysaccharide having a high methoxyl content; spin-coating the polysaccharide onto a substrate to form a thin film; and exposing the thin film polysaccharide to a concentration of CaCl₂ in ethanol to produce a crosslinked coating. At least one cell may be adhered to the thermo-responsive crosslinked coating on the substrate and changing cell culture temperature may change the adherence level of the at least one cell to the thermo-responsive crosslinked coating on the substrate.

The polysaccharide may be pectin. The at least one cell may be incubated at the cell culture temperature of about 37° C. to adhere the at least one cell to the thermo-responsive crosslinked coating. The cell culture temperature may be lowered to about 20° C. detaches the at least one cell from the thermo-responsive crosslinked coating.

### BRIEF DESCRIPTION OF THE DRAWINGS

For a fuller understanding of the invention, reference should be made to the following detailed description, taken in connection with the accompanying drawings, in which:

FIG. 1 is an image depicting ATR-FTIR spectra of coatings of pectin cross-linked in ethanol at different CaCl₂ concentrations. For comparison purposes, the ATR-FTIR spectrum of a coating before cross-linking is also shown.

FIG. 2A-B are a series of images depicting dependence of (A) the wavenumber value of the —COO⁻ peak and (B) the area of the (COO⁻/COOR) peak of pectin coatings cross-linked in ethanol.

FIG. 3A-B is a series of images depicting dependence of the wavenumber values of (a) —CH₃ and (b) —COOR of cross-linked pectin networks at different CaCl₂ concentrations.

FIG. 4 is an image depicting ATR-FTIR spectrum of (a) dried pectin, pectin networks cross-linked by CaCl2 at 1 M at (b) t=0 min, (c) t=20 min, (d) t=40 min, and (e) t=60 min.

FIG. 5 is an image depicting swelling ratios (H/Hd_{dry}) of cross-linked pectin networks at different CaCl₂ concentrations in contact with water.

FIG. 6A-B is a series of images depicting AFM height images (2 μm×2 μm) of (a) dry pectin coating before exposure to CaCl₂ and (b) dry pectin coating after cross-linking with CaCl₂.

FIG. 7A-B are a series of images depicting swelling ratios (H/H_{dry}) of (A) P-CaCl₂ 1 M, (B) P-CaCl₂ 0.0001 M, in contact with water, and a buffer solution at pH values of 2 and 9.

FIG. 8 is an image depicting ATR-FTIR spectra of pectin-CaCl₂ 0.1M in water at (a) 25° C., (b) 37° C. (c) 45° C. (d) 55° C. (spectra of water are subtracted for all samples), and (e) dried sample.

FIG. 9 is an image depicting progression of normalized wavenumber f for the —CH₃ and —COO⁻ groups as a function of the average pectin volume fraction ϕ.

FIG. 10 is an image depicting ATR-FTIR spectra of pectin-CaCl₂ 0.1M in (a) H2O at 55° C., and (b) D2O at 55° C.

FIG. 11A-B are a series of images depicting NIH3T3 fibroblast cells on a glass coverslip coated with cross-linked highly-methoxylated pectin at (a) 37° C. and (b) 20° C. after t=60 min of incubation; scale bar=100 μm (460-500 nm excitation wavelength).

FIG. 12A-D are a series of images depicting NIH3T3 fibroblast cells on a glass coverslip at (a) 37° C. (phase contrast micrographs), (b) 37° C. (fluorescence micrographs), (c) 20° C. after t=60 min of incubation (phase contrast micrographs), and (d) 20° C. after t=60 min of incubation (fluorescence micrographs); scale bar=100 μm.

FIG. 13 is an image depicting the swelling response of pectin coatings. As shown in the image, the swelling response is temperature dependent with the coating being cell repellant at 25° C. and cell adhesive at 37° C.

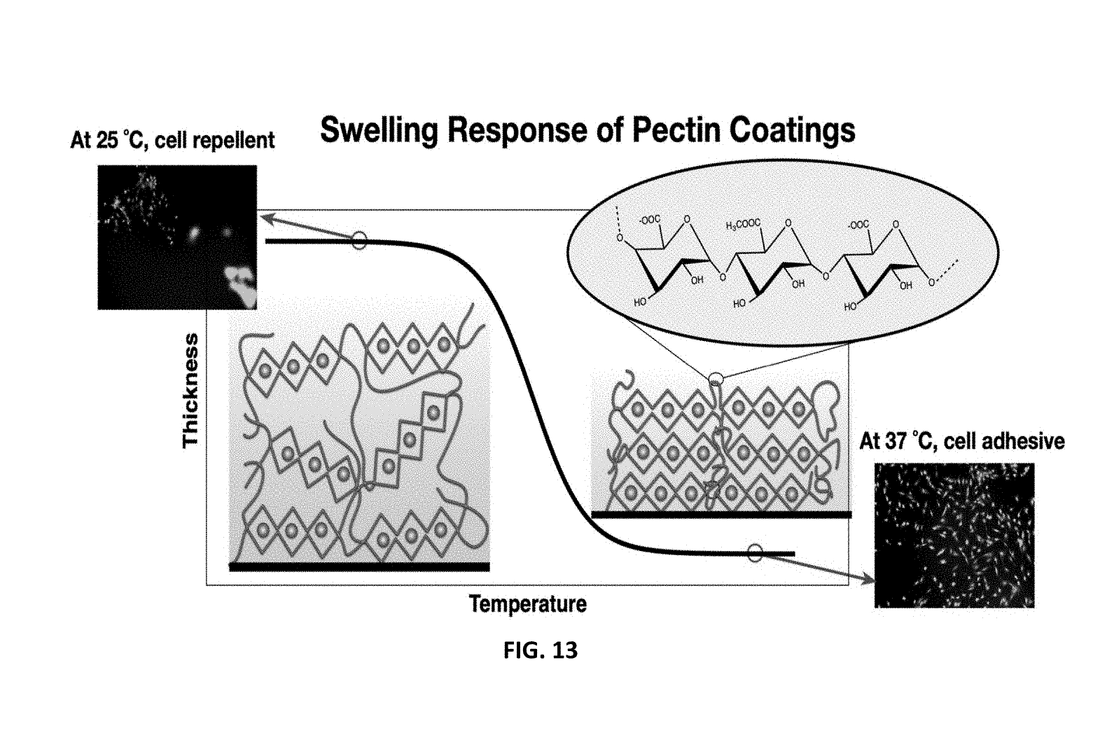

FIG. 14A-F are a series of images depicting cutin surface. (A) low magnification scan of cross-section of cutin surface in the Opuntia ficus-indicia cactus. Scale bar, 100 μm; (B) low magnification scan of top view of cutin surface in the Opuntia ficus-indicia cactus. Scale bar, 100 μm; (C) medium magnification scan of top view with zoom in on cutin layers; (D) top view zoom in on cutin layers of (C); (E) high magnification scan taken by Dr. David Webb and adapted from public domain, Scale bar, 200 μm; (F) high magnification scan taken by Dr. David Webb and adapted from public domain, scan size is ca. 80 μm. The smooth part is the cuticle part of the plant formed by cutin.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

Definitions

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supercedes any disclosure of an incorporated publication to the extent there is a contradiction.

All numerical designations, such as pH, temperature, time, concentration, and molecular weight, including ranges, are approximations which are varied up or down by increments of 1.0 or 0.1, as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

Where a range of values is provided, it is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed in the invention. The upper and lower limits of these smaller ranges may independently be excluded or included within the range. Each range where either, neither, or both limits are included in the smaller ranges are also encompassed by the invention, subject to any specifically excluded limit in the stated range. Where the stated range includes one or both of the limits, ranges excluding either or both of those excluded limits are also included in the invention.

The term “about” or “approximately” as used herein refers to being within an acceptable error range for the particular value as determined by one of ordinary skill in the art, which will depend in part on how the value is measured or determined. As used herein, the term “about” refers to ±10%.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

Concentrations, amounts, solubilities, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include the individual values and sub-ranges within the indicated range. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range or the characteristics being described.

As used in the specification and claims, the singular form “a”, “an” and “the” include plural references unless the context clearly dictates otherwise. For example, the term “a nanoparticle” includes a plurality of nanoparticles, including mixtures thereof.

The terms “thin film”, “coating”, or “thin film coating” are used interchangeably herein to refer to at least one layer of the polysaccharide, such as pectin polysaccharide, or cutin that is placed over at least a portion of the surface of a substrate.

The term “substrate” as used herein refers to a base surface onto which the thin film or coating is applied. In some embodiments, the substrate may be a glass slide onto which the thin film or coating is applied. In this embodiment, at least one cell may subsequently be applied to the surface of the thin film or coating so that the at least one cell is supported on the substrate by the thin film or coating.

The term “polysaccharide” as used herein refers to a polymeric carbohydrate molecule comprised of long chains of monosaccharide units bound together by glycosidic bonds. Polysaccharides used herein can be heteropolysaccharides or homopolysaccharides.

The term “heteropolysaccharide” as used herein refers to a polysaccharide comprised of at least two different monosaccharides. Exemplary heteropolysaccharides used herein include, but are not limited to, pectin.

The term “homopolysaccharide” as used herein refers to a polysaccharide comprised of only one type of monosaccharide.

The term “monosaccharides” as used herein refers to monomeric units of simple sugars such as glucose, fructose and galactose.

The term “pectin” as used herein refers to a water soluble heteropolysaccharide found in the cell walls and intercellular tissues of certain plants such as cactus and fruit plants.

The term “high methoxyl content” as used herein refers to a polysaccharide, in particular a pectin polysaccharide, having ≥50% esterification.

The term “low methoxyl content” as used herein refers to a polysaccharide, in particular a pectin polysaccharide, having <50% esterification.

The term “cutin” as used herein refers to a waxy waterproof substance, consisting of derivatives of fatty acids as well as possible soaps and resinous materials. Natural cutin is the main constituent of the cuticle of plants. Synthetic cutin can be manufactured using cutin precursors or monomers.

The term “natural” as used herein refers to a product existing in nature. In particular, “natural” refers to cutin or polysaccharides, such as pectin, extracted from a plant species for use in manufacturing a responsive coating. “Naturally derived” refers to use of some form of a product that was originally found in nature.

The term “synthetic” or “synthetically derived” as used herein refers to a product produced artificially by human hand by chemical synthesis. In particular, “synthetic” refers to the manufacture of a product which mimics a natural product. Both naturally and synthetically derived products are contemplated for use herein.

### EXAMPLE 1

### Pectin Polysaccharides

One strategy for preparing thin coatings is to first cross-link pectin in an aqueous solution in same manner as preparing bulk gels, and then to post-process the gel into a coating. A more direct strategy, however, is to cross-link in situ a preformed coating of pectin. The latter is preferable to post-processing of a bulk gel in that it allows better control over both thickness and cross-link density. Coatings prepared in this manner display properties distinct from bulk prepared gels, as is common for gels prepared at surfaces and interfaces. (Mateescu, A.; Wang, Y.; Dostalek, J.; Jonas, U., Thin hydrogel films for optical biosensor applications. Membranes 2012, 2 (1), 40-69; Stuart, M. A. C.; Huck, W. T.; Genzer, J.; Muller, M.; Ober, C.; Stamm, M.; Sukhorukov, G. B.; Szleifer, I.; Tsukruk, V. V.; Urban, M., Emerging applications of stimuli-responsive polymer materials. Nature materials 2010, 9 (2), 101-113; Lin, G.; Chang, S.; Kuo, C. H.; Magda, J.; Solzbacher, F., Free swelling and confined smart hydrogels for applications in chemomechanical sensors for physiological monitoring. Sensors and Actuators B: Chemical 2009, 136 (1), 186-195).

The inventors have developed a method for cross-linking coatings of pectin with CaCl₂. Pectin was first spin-casted onto a solid surface followed by incubation in a CaCl₂/ethanol solution. Ethanol, a poor solvent for pectin, prevents dissolution of the coating while simultaneously allowing CaCl₂ diffusion to establish Ca²⁺ bridges between carboxyl groups. It was found that pectin polysaccharides with 67-70% esterification could be cross-linked in this manner, despite the absence of gelation in analogous aqueous solutions at concentrations near the solubility limit of the pectin polysaccharide. Moreover, these coatings exhibited a drastic change in solubility with temperature, similar to so-called “thermoresponsive” polymers that have a lower critical solution temperature (LCST). (M. Shibayama, T. Tanaka, Volume Phase Transition and Related Phenomena of Polymer Gels, Responsive Gels: Volume Transitions I, Springer, 1993, pp. 1-62).While such behavior has not been observed in bulk gels of pectin, it presumably results from the difficulty associated with cross-linking highly esterified pectins with calcium. As used herein, ethanol, by preventing dilution of the pectin chains, allows sufficient proximity between adjacent non-esterified regions for calcium to form stable cross-link zones.

In light of these findings, high-methoxyl pectin coatings were crosslinked over a range of CaCl₂ concentrations in ethanol, and their swelling behavior in contact with water was characterized with both ellipsometry and ATR-FTIR. At temperatures below approximately 35° C., the coatings were hydrophilic and absorbed water. At higher temperatures, the coatings expelled water and collapsed giving rise to distinctive de-swelling profiles. The de-swelling profiles were studied as a function of various stimuli to elucidate the key factors governing the swelling behavior and to better understand the hydrophobic/hydrophilic transition. FTIR measurements suggest that the esterified galacturonic acid residues are hydrated at lower temperatures and that the hydrophilic/hydrophobic transition is driven by dehydration of the esterified galacturonic acid residues as the temperature is increased. Moreover, by adjusting the degree of cross-linking and pH of the surroundings, it was shown that the thermal response of the pectin coatings could be carefully controlled.

It is well-known that thermally-responsive polymer coatings, especially poly(N-isopropylacrylamide), can be used as cell culture platforms for cell release applications. Considering that the thermal response of cross-linked high-methoxyl pectin coatings is similar to poly(N-isopropylacrylamide), the inventors investigated the possibility of employing such coatings for cell culture and release. Cultured fibroblast cells were seeded and proliferated on coatings of cross-linked high methoxyl pectin at 37° C., which could be subsequently released at 20° C. These results demonstrate the potential of naturally occurring polysaccharides for tunable surfaces and coatings.

Characterization of the Surface-Attached Thin Films of Cross-Linked Pectin using ATR-FTIR

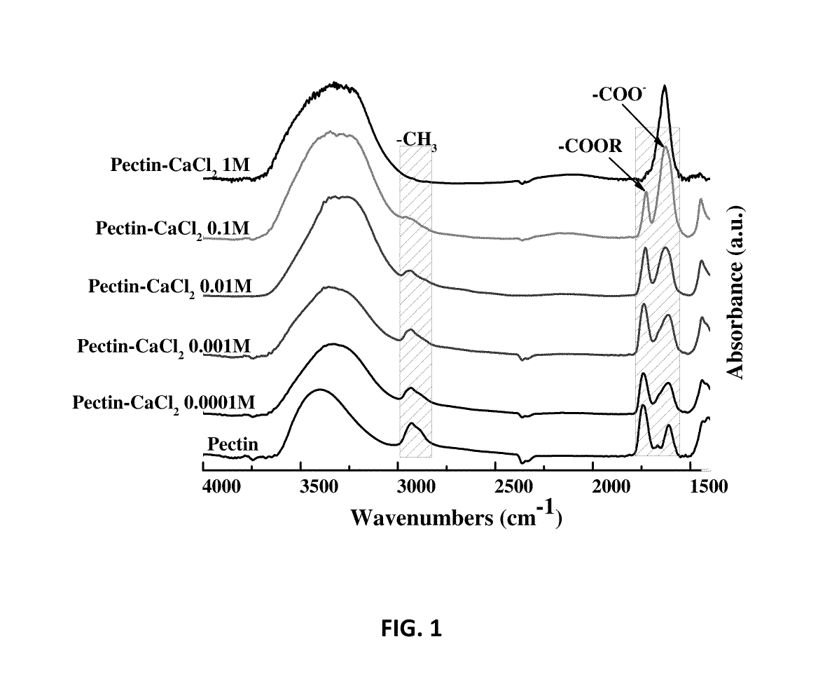

ATR-FTIR spectroscopy was used to assess the extent of cross-linking of the pectin as well as the molecular interactions between pectin and Ca²⁺ ions by monitoring the carboxyl and carbomethoxy groups. (Patra, L.; Messman, J. M.; Toomey, R., On the nature of volume-phase transitions in photo-cross-linked poly (cyclopropylacrylamide) and poly (N-vinylisobutyramide) coatings. Soft Matter 2013, 9 (16), 4349-4356; Synytsya, A.; Čopiková, J.; Matějka, P.; Machović, V., Fourier transform Raman and infrared spectroscopy of pectins. Carbohydrate Polymers 2003, 54 (1), 97-106). FIG. 1 compares the FTIR spectra of an as-cast dry citrus pectin coating (before exposure to calcium) with the FTIR spectra of dry pectin coating after cross-linking with CaCl₂ in ethanol. All spectra show a broad peak at approximately 3000-3600 cm⁻¹ corresponding to absorption due to the stretching vibration of hydroxyl (—OH) groups along the pectin backbone. The band at ˜2920-2950 cm⁻¹ corresponds to the methyl (—CH₃) vibration in the esterfied galacturonic acid residues. The bands at ˜1740-1750 cm⁻¹ and ˜1625-1640 cm⁻¹ correspond to the carboxy group in the esterfied residue (—COOR) and acid salt residue (—COO⁻), respectively. (A. Balaria, S. Schiewer, Assessment of biosorption mechanism for Pb binding by citrus pectin, Sep. Purif. Technol. 63 (2008) 577-581). Wavenumbers smaller than ˜1500 cm-1 are considered as the fingerprint regions of the spectra revealing information regarding the absorption of C—C, C—O—C, and C—OH stretching vibration modes as well as the glycosidic linkage vibrations. Such wavenumbers are not typically used to probe the environment of functional groups due to the presence of multiple overlaying absorption bands and hence are not shown in FIG. 1. (J. Thibault, M. Rinaudo, Chain association of pectic molecules during calcium-induced gelation, Biopolymers 25 (1986) 455-468).

The changes in the alkyl (—CH₃) and carboxyl-ester (—COOR) peaks with cross-linking reflect changes in the environment surrounding the carbomethoxy groups while changes in the carboxylate (—COO⁻) peaks probe the COO⁻—Ca⁺² interaction. Interestingly, FIG. 1 shows a pronounced increase in the area of −COO⁻ vibration relative to the other vibrations.

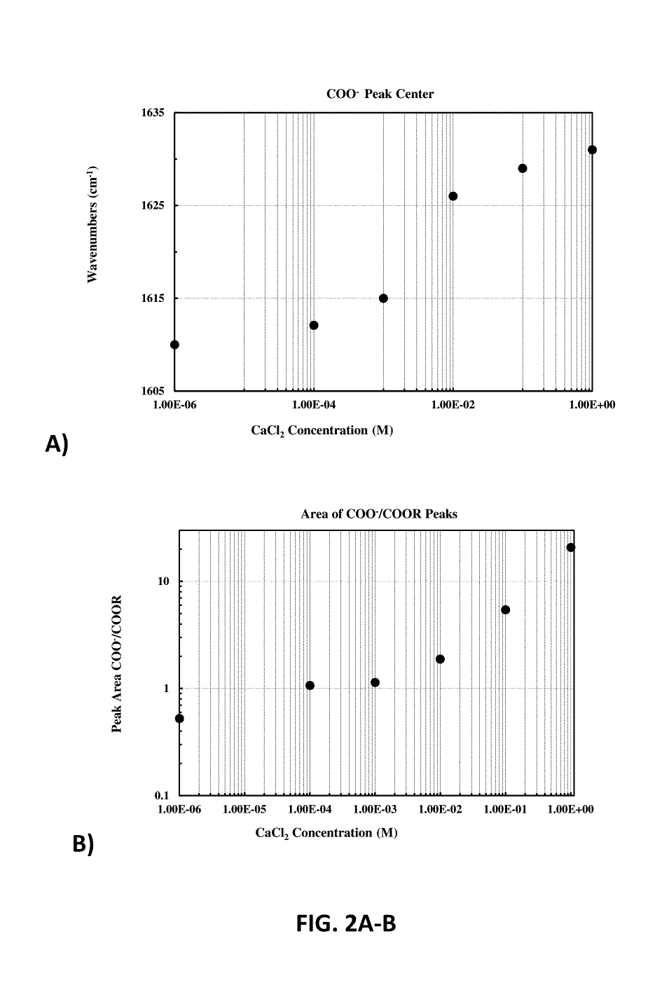

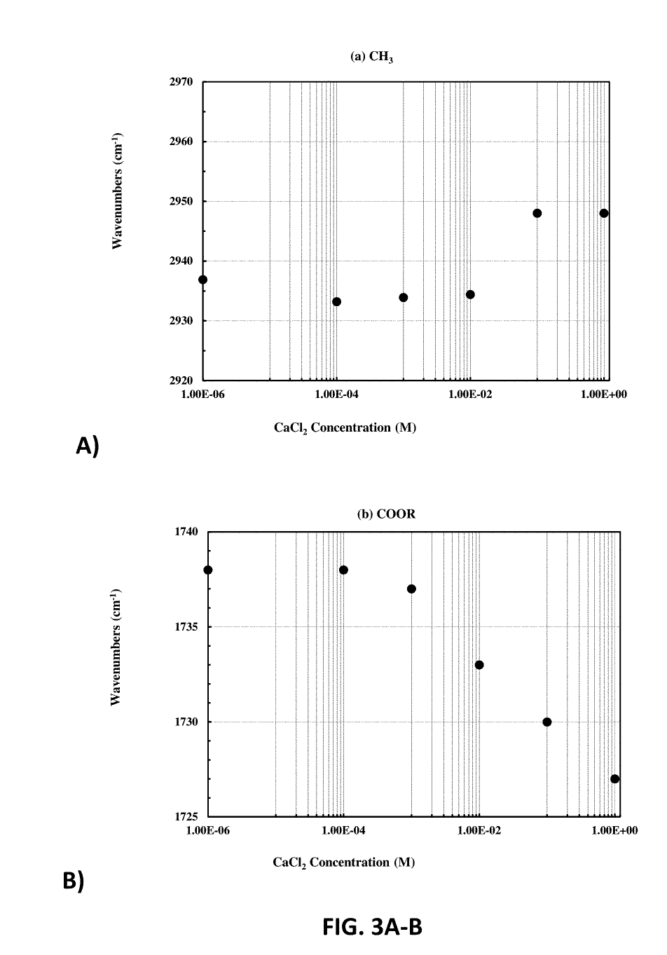

The spectra presented in FIG. 1 reveal both a significant shift in the wavenumber of the —COO⁻ peak as well as an increase in its area relative to the other peaks with increasing CaCl2 concentration. FIG. 2(a) shows the wavenumber of the —COO—group versus CaCl2 concentration, which shifts from 1610 cm⁻¹ in pure pectin to 1631 cm⁻¹ (Δv_{—COO}_{^{−}}=21 cm⁻¹) at 1M CaCl₂. Wavenumber shifts also occur for the bands associated with the —CH3 and carboxy ester —COOR groups, however they are not as significant as the —COO⁻ group (FIG. 3). Cross-linking of pectin using CaCl₂ resulted in the shift of alkyl (—CH₃) band from 2937 cm⁻¹ to 2948 cm⁻¹(Δv_{—CHn}=11) for pectin and pectin networks cross-linked by CaCl₂ at 1 M respectively. The absorption band associated with the carboxyl-ester (—COOR) groups red-shifted from 1738 cm⁻¹ to 1727 cm⁻¹ (Δv_{—COOR}=11) for pectin and pectin networks cross-linked by CaCl₂ at 1 M respectively. Such observations are consistent with bulk cross-linked gels in water indicating that cross-linking in ethanol does not appreciably affect the relative shifts of the infrared vibrations. (A. Assifaoui, C. Loupiac, O. Chambin, P. Cayot, Structure of calcium and zinc pectinate films investigated by FTIR spectroscopy, Carbohydr. Res. 345 (2010) 929-933; H.-A. Tajmir-Riahi, Sugar complexes with calcium ion: infrared spectra of crystalline D-glucuronic acid and its calcium complexes, Carbohydr. Res. 122 (1983) 241-248). It should be noted, however, that CaCl₂ does not fully dissociate into free ions in pure ethanol, and that cross-linking of the coatings is likely mediated by trace amounts of water associated with the pectin polysaccharide. (A. Bald, A. Szejgis, J. Barczyńska, H. Piekarski, Effect of ionic association on the B coefficient for CaCl2 in ethanol-water mixtures at 298.15 K, Phys. Chem. Liquids 37 (1999) 125-135). As a result, most of the dissolved CaCl₂ is unavailable for cross-linking, which may explain why changes in both the —COO⁻ peak position as well as the increase in its area relative to the other peaks are observed between 0.1M-1.0M CaCl2, well above the expected saturation limit of Ca²⁺ ions for the coatings.

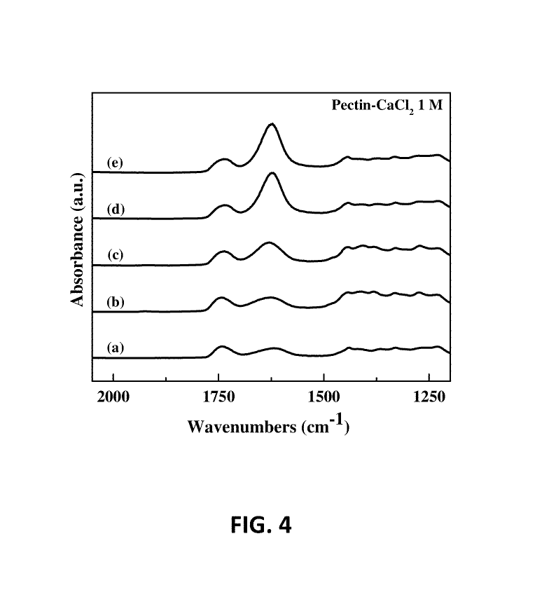

Of particular note is the enhancement in the absorption associated with the COO⁻ vibration between 1625-1640 cm⁻¹ with increasing CaCl₂ concentration. The ratio of peak area of the acid to the ester group as a function of calcium concentration is presented in FIG. 2(b). Such enhancement has also been observed in bulk cross-linked gels. (S. Seslija, D. Veljovic, M. K. Krusic, J. Stevanovic, S. Velickovic, I. Popovic, Crosslinking of highly methoxylated pectin with copper: the specific anion influence, New J. Chem. 40 (2016) 1618-1625). To understand the source of this enhancement, the ATR-FTIR spectrum of a pectin coating cross-linked at 1M CaCl₂ was recorded over time (FIG. 4). The overall area associated with the —COOR band remained relatively unchanged while the overall area of the —COO⁻ band increased significantly. The increase in the area under the —COO⁻ is most likely related to the displacement of the monovalent counterion with Ca²⁺, which alters the dipole moment and intensity of the absorption band. (B. C. Smith, Infrared Spectral Interpretation: a Systematic Approach, CRC press, Boca Raton, 1998).

Phase Transition Behavior of Surface-Attached Gel Films as a Function of Temperature in Contact with Aqueous Solutions

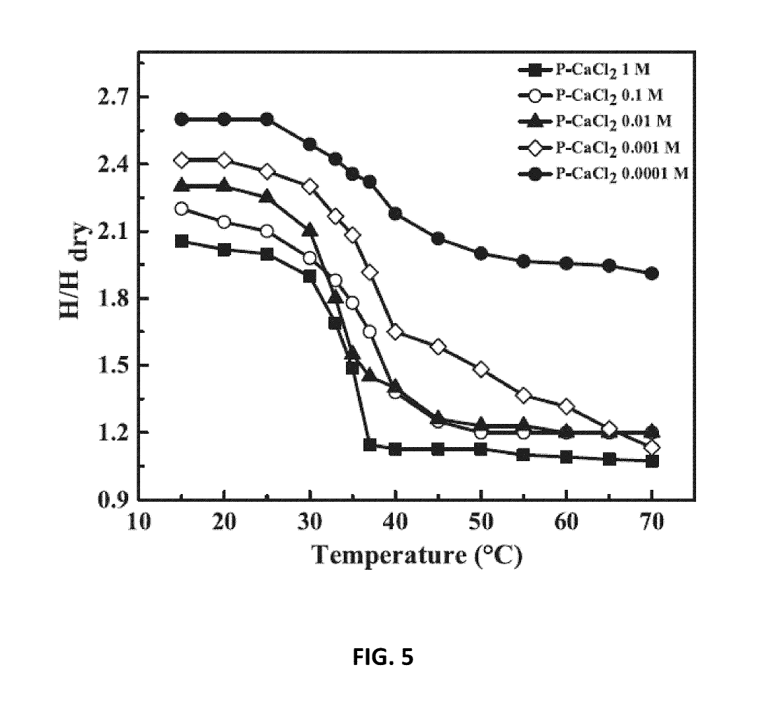

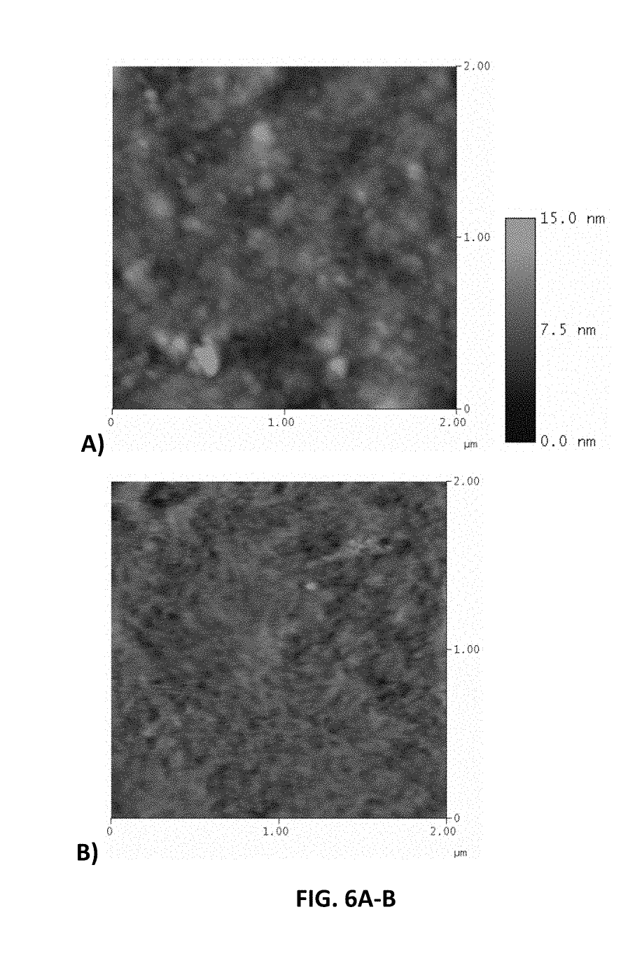

The swelling profiles of cross-linked pectin films were characterized by ellipsometry to monitor the average water content in the networks as a function of both temperature and cross-linking. ATR-FTIR was used in parallel with ellipsometry to provide information regarding the environment surrounding the —CH₃, —COOR, and —COO⁻ groups. The swelling ratios (H/Hdry) that represent the best fits to the experimental values are shown in FIG. 5. The dry thickness of each film was approximately 80 nm as measured by ellipsometry. Moreover, the surface topography and roughness of the dry pectin coating before exposure to calcium and the dry pectin coating after cross-linking with CaCl₂ were investigated using AFM (FIG. 6). AFM images of the dry films displayed relatively smooth surfaces with roughness values of less than 5 nm.

The temperature of the films in contact with water was varied at a rate of 0.5° C. per minute and samples were maintained at each temperature for 20 minutes to ensure equilibrium. The equilibrium water content and the resultant thickness strongly depended on temperature. All pectin coatings followed a similar trend, wherein the coatings swelled to approximately 2-3 times the dry thickness at temperatures below 30° C. In this regime, the degree of swelling depended somewhat, albeit weakly, on the CaCl₂ concentration used to cross-link the coating. At the lowest CaCl₂ concentration (10⁻⁴ M), the coating swelled up to 2.6 times the dry layer thickness. As the CaCl2 concentration is increased to 1 M, the coating swelled to 2.1 times its dry layer thickness, a reduction of only 20%.

As the temperature is increased above 30° C., the coatings expelled water and started to contract, which depended strongly on the CaCl₂ concentration used to cross-link the coating. At the lowest concentration of 10⁻⁴ M, the coating gradually expelled water between 30° C. and 50° C. until the degree of swelling reached a plateau that was approximately twice the dry layer thickness. As the CaCl₂ concentration was increased, the transition zone became narrower and sharper. At 1M CaCl_{2,} the highest concentration, the coating collapsed to 1.1 times the dry layer thickness. In no cases was the water completely expelled even as the temperature was increased up to 70° C.

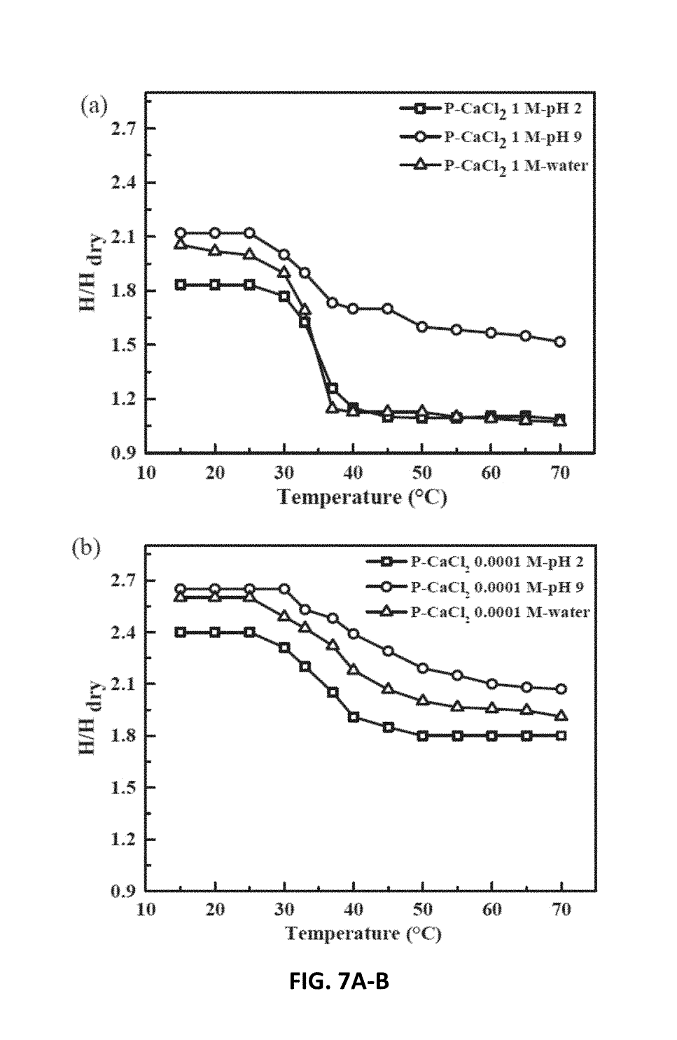

To investigate the effect of pH on the swelling behavior of the crosslinked pectin films, the coatings were contacted with a buffer solution at a pH of 2 (potassium chloride-hydrochloric acid buffer) and 9 (boric acid-hydrochloric acid buffer) for both the high and low cross-link densities. These pHs where chosen since the pKa of pectin is near 3.5. (Sriamornsak, P., Chemistry of pectin and its pharmaceutical uses: A review,” Silpakorn University International Journal 3.1-2 (2003): 206-228). FIG. 7(a) compares the effect of pH on a high cross-linked coating (1M CaCl₂) that collapsed in unbuffered water and FIG. 7(b) compares the effect of pH on a low cross-linked coating (10⁻⁴ M CaCl₂) that only partially deswelled in unbuffered water. The swelling trends for both coatings depended on pH, but the effect was also generally small.

In the case of the highly cross-linked coating, the thickness of the coating in unbuffered water was between the thickness values at pH=2 and pH=9 in the low temperature regime (below 30° C.). At a pH of 9, the coating expanded by about 2%, and at a pH of 2, the coating contracted by approximately 10%. It is expected that an increase in pH shifts the COOH—COO⁻ balance to primarily —COO⁻ around the pKa, maximizing negative charges along the pectin chain. Hence, the relatively small change in the degree of swelling between unbuffered water and pH 9 suggests that the galacturonic acid residues are primarily in their charged (or carboxylate) state under neutral conditions. As the temperature was increased above the volume-phase transition temperature for the coating in unbuffered water, the coating at pH=9 did not completely collapse, which points to a delicate balance between charged and uncharged residues in controlling the transition behavior. As the pH is reduced to 2, these groups convert to the acid form slightly condensing the layer. This effect is small, nevertheless, at temperatures below 30° C. At higher temperatures, the behavior becomes more complex and depends on the cross-link density. That said, increasing the pH to 9 can significantly enhance degree of swelling. At the highest cross-link density (1 M CaCl₂), the degree of swelling above 30° C. increases almost 50% in comparison to unbuffered water.

In the case of the low cross-linked coating, similar to the high crosslinked coating, the thickness of the coating in unbuffered water was also between the values at pH=2 and pH=9; however, the same overall trend persisted over the entire temperature range. At 10⁻⁴ M CaCl2, the degree of swelling also increases, although to a lower extent (˜5%) as the pectin coating remains highly hydrated even in unbuffered water. Moreover, the effect was relatively small (5-10%), which develops a picture that changes in ionization has only a small effect unless hydrophobic interactions can successfully compete with hydrophilic interactions.

Role of Water in Structural Changes of Crosslinked Coatings During De-Swelling as Studied by FTIR

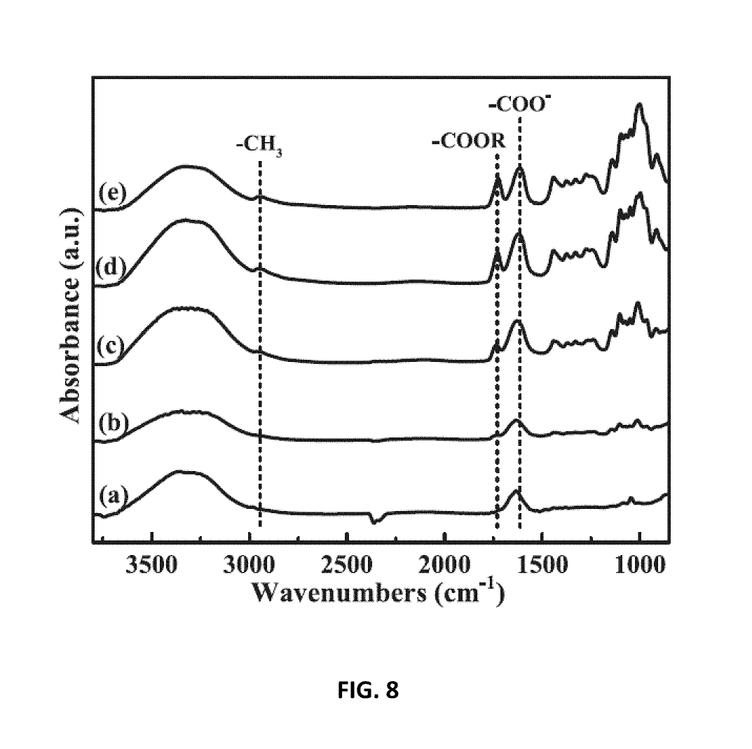

To understand the origin of this competition between hydrophilic and hydrophobic interactions, ATR-FTIR was used to investigate the changes in the environment surrounding the —CH₃, —COOR, and —COO⁻ groups of the pectin coatings as a function of temperature in contact with unbuffered water. First, the coatings were heated to 80° C. to remove water. Following dehydration of the coatings, immersing the coatings in water at 25° C. produced two noticeable peaks at 3300 cm⁻¹ and 1636 cm⁻¹ corresponding to the stretching and bending vibrations of water, which generally masked the relevant frequencies in the pectin coating. Therefore, to help in analysis, the water spectrum was subtracted from the hydrated coatings at the relevant temperature, which revealed functional groups in the hydrated polymer and reduced the interference of bulk water. (J. Dybal, M. Trchová, P. Schmidt, The role of water in structural changes of poly (Nisopropylacrylamide) and poly (N-isopropylmethacrylamide) studied by FTIR, Raman spectroscopy and quantum chemical calculations, Vib. Spectrosc. 51 (2009) 44-51). Difference spectra of the pectin coatings for select temperatures are shown in FIG. 8 along with an FTIR spectrum of a dry pectin coating. The dashed lines correspond to the wavenumber of the —CH3, —COOR, and —COO⁻ groups in the dry state. In water at 25° C., all group wavenumbers are blue-shifted with respect to the dry values. This blue shift can be explained, to first order, within the context of the Kirkwood-Bauer-Magat (KBM) equation, which describes solvent-induced shifts of wavenumber based on the dielectric constant of the solvent. (W. West, R. T. Edwards, The infrared absorption spectrum of hydrogen chloride in solution, J. Chem. Phys. 5 (1937) 14-22; J. G. Kirkwood, Theory of solutions of molecules containing widely separated charges with special application to zwitterions, J. Chem. Phys. 2 (1934) 351-361). The KBM equation predicts that as the dielectric constant of the solvent is increased, there is a subsequent shift to higher wavenumbers. The shifts in FIG. 8, therefore, can be interpreted by the change in the dielectric constant due to solvation by water. That said, the KBM equation, does not include specific solute-solvent interactions (such as hydrogen bonding which can lead to a redshift in wavenumber), and therefore the wavenumbers can only serve in a qualitative understanding of the hydration state. (Q. Liu, J. Zheng, D. Fang, Solvent effects on infrared spectra of methyl methacrylate, Spectrosc. Lett. 37 (2004) 225-233).

Upon heating the coatings from 25° C. up to 37° C., alkyl (—CH₃) band slightly red-shifted. The same pattern was detected for the peak at 1636 cm⁻¹. Further heating of the coatings above 37° C. resulted in the appearance of three distinct bands associated with carboxyl-ester (—COOR), carboxylate (—COO⁻) and alkyl (—CH₃) groups (i.e. peaks at 1733 cm⁻¹, 1628 cm⁻¹ and 2962 cm⁻¹ at 45° C.), consistent with the de-swelling of the pectin coatings as measured with ellipsometry (i.e. the density of groups increases next to the crystal surface)⁴⁵. With further heating the coating from 45° C. up to 55° C., the alkyl (—CH₃) band red-shifted from 2962 cm⁻¹ to 2955 cm⁻¹ which is nearly the same (Δv_{—CHn}=2) compared to the measured values at the dried state. The carboxyl-ester (—COOR) band also shifted to its measured value at the dried state (Δv_{-COOR}=3), suggesting that at this temperature, these two functional groups are nearly dehydrated. On the other hand, the carboxylate (—COO⁻) groups also red-shifted from 1628 cm⁻¹ to 1621 cm⁻¹ which is still 7 cm⁻¹ higher compared to the peak value at the dried state, suggesting that water remains bound to the carboxylate groups. The ellipsometry results showed that at this temperature, the coatings are at their weakly swollen states and do not completely expel water.

Upon heating the coatings from 25° C. to 55° C., all three group wavenumbers (—CH3, —COOR, and —COO⁻) red-shifted towards the dry state values. To aid in analysis, a normalized wavenumber f can be defined as

$$f = \frac{v ( T ) - {v}_{dry}}{v ( {T}_{0} ) - {v}_{dry}}$$

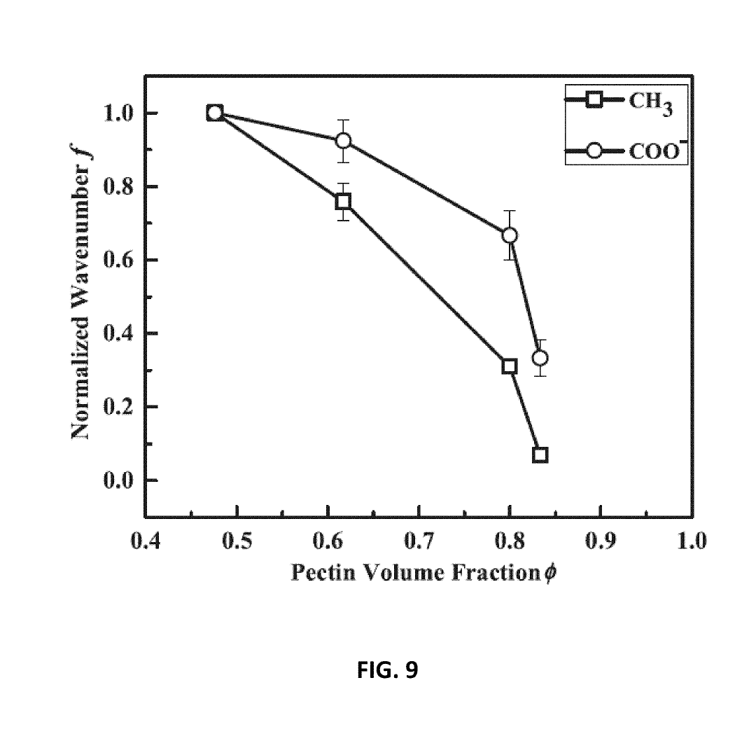

for each functional group, where v (T) is the associated wavenumber at a temperature T, vdry is the wavenumber in the dry state, and v (To) is the wavenumber at 25° C. FIG. 9 shows the progression of f for both the —CH3 and —COO⁻ groups as a function of ϕ, where

$$φ = \frac{{H}_{dry}}{H ( T )} ,$$

is the average pectin volume fraction of the coating as determined from ellipsometry. Between 25° C. and 45° C. (corresponding to values of ϕbetween 0.48 to 0.8), there is an almost linear reduction in f from 1.0 to 0.31 for the —CH3 groups and from 1.0 to 0.67 for the —COO⁻ groups. In other words, f decreases by 69% for the —CH3 groups but only by 33% for the —COO⁻ groups over the same change in degree of swelling. As temperature is further increased to 55° C., the average pectin volume fraction ϕ of the coating increases only from 0.8 to 0.83, yet the value off for the —CH3 groups drops to nearly zero (0.07), signifying that the wavenumber is approximately identical to its dry state value. The value off for —COO⁻, however, remains 33% above its dry state value.

Evaluation of f as a function of ϕ, therefore, suggests the heterogeneous dehydration of the pectin coating as driven by the methyl groups. Similar dehydration was also observed for the —COOR vibration, however, the exact position of the vibration could not be reliably determined at 25° C. and so was excluded from the analysis. Such relative changes in the wavenumbers, therefore, imply that the collapse of the pectin network is driven by hydrophobic interactions amongst the esterified acid residues in a similar fashion as to how the isopropyl group drives the volume-phase transition of poly(N-isopropylacrylamide), or poly(NIPAAM), a well-known and well-studied LCST polymer. (M. Heskins, J. E. Guillet, Solution properties of poly (N-isopropylacrylamide), J. Macromol. Sci.—Chem. 2 (1968) 1441-1455; T. Tanaka, Phase transitions in gels and a single polymer, Polymer 20 (1979)1404-1412; H. G. Schild, Poly (N-isopropylacrylamide): experiment, theory and application, Prog. Polym. Sci. 17 (1992) 163-249). Poly(NIPAAm) is comprised of units containing a hydrophilic amide and a hydrophobic isopropyl group. Analogous to the findings herein, the isopropyl groups of cross-linked coatings of poly (NIPAAm) blue-shift upon swelling at low temperatures and return to the dry-state value at temperatures above the volume-phase transition. This same study showed that the hydrophilic amide groups, however, do not completely return to their dry state values even at temperatures far above the volume-phase transition, suggesting water remained bound to the amide groups. (A. Vidyasagar, H. L. Smith, J. Majewski, R. G. Toomey, Continuous and discontinuous volume-phase transitions in surface-tethered, photo-crosslinked poly (Nisopropylacrylamide) networks, Soft Matter 5 (2009) 4733-4738).

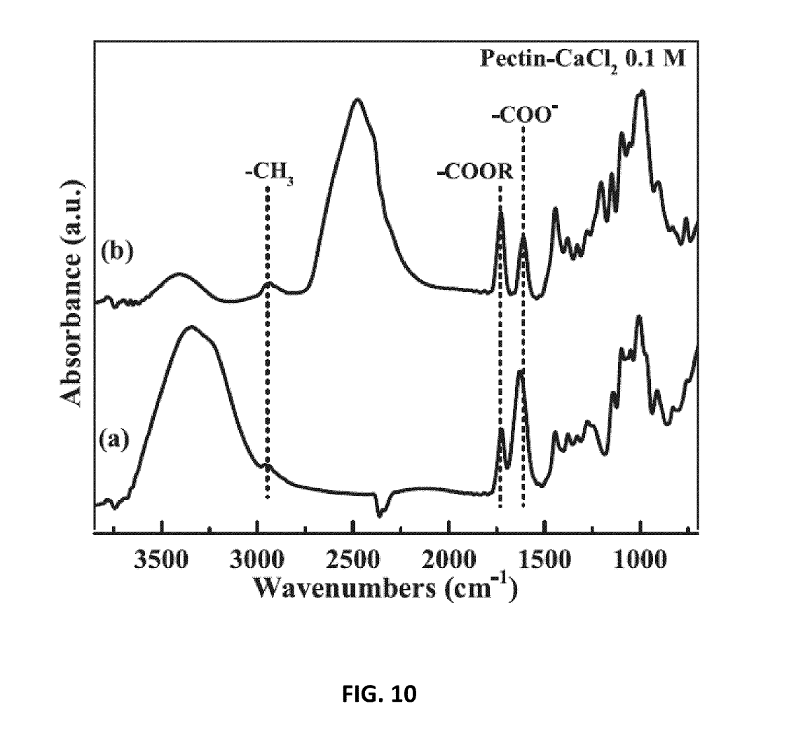

As temperature is increased, it is therefore anticipated that the pectin coating collapses in a heterogeneous state with —COOR rich domains where water is excluded and —COO⁻ rich domains where water remains bound. To test this hypothesis, D2O was exchanged with H₂O while the coating was at 55° C. FIG. 10 shows the FTIR spectra of coatings before and after this exchange. Consistent with the hypothesis, the peak associated with —COO⁻ groups red-shifted by 9 cm⁻¹ as H2O is replaced by D2O. This suggests that the carboxylate groups (hydrophilic rich domains) are surrounded by water molecules at 55° C., as evidenced by the fact that D2O can be readily exchanged by H2O. However, the peak positions associated with the —CH3 and —COOR were invariant to the H2O/D2O exchange, suggesting that the hydrophobic rich domains are nearly dehydrated at 55° C.

As a demonstration of the thermal response of the pectin coatings, temperature-induced changes in cell morphology and spreading area were investigated. NIH3T3 mouse embryonic fibroblast cells were seeded on both bare glass coverslips (as control) and glass coverslips coated with cross-linked (0.1M CaCl₂). The coatings were imaged after 48 h of incubation at 37° C. At least 3 images per sample were acquired and approximately 300 cells were analyzed per experiment.

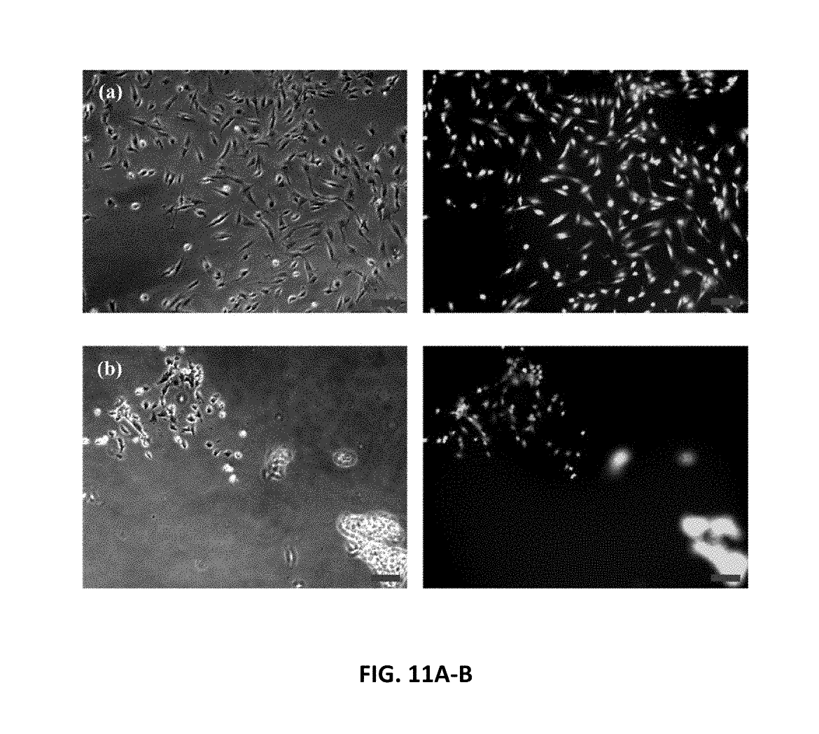

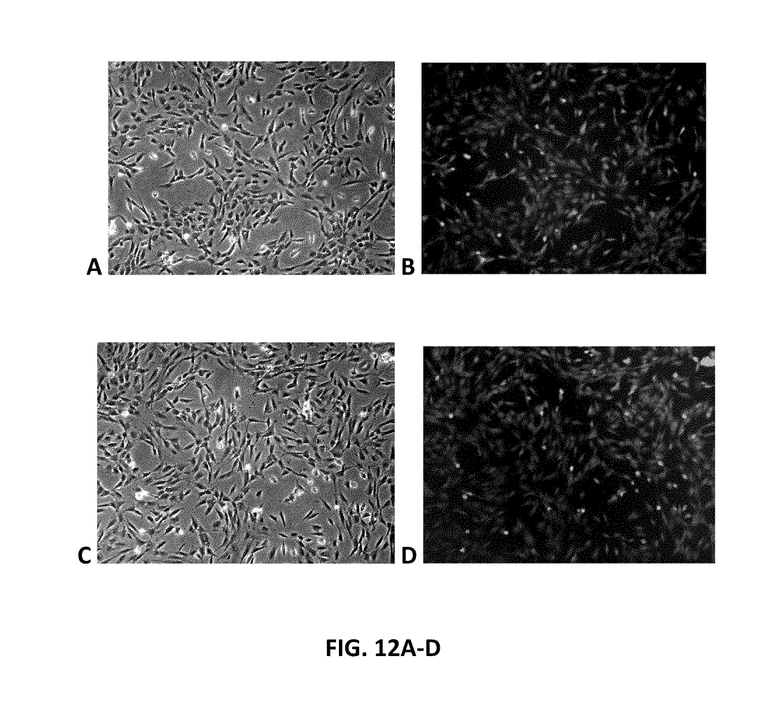

Cell spreading area and morphology were found to be nearly similar in the coated and control dishes after 48 h of incubation at 37° C. Upon lowering the culture temperature to 20° C., attached cells underwent morphological changes and started to contract but only on the pectin coated glass coverslips. The cell morphology changed from a spread structure to a more rounded shape after only 5 min of incubation. After 60 min of incubation at 20° C., the majority of the cells detached from the pectin coatings, as shown in FIG. 11. In contrast, in the case of cells cultured on the glass coverslips, no significant differences were found either in the cell spreading or cell morphology after 60 min at an incubation temperature of 20° C. (See FIG. 12).

The change in cell morphology and decreased spreading is attributed to the increased hydration and swelling of the coatings upon temperature reduction. The contact angle of the cross-linked (0.1M CaCl₂) pectin coating at 37° C. was 62°±1°. At 37° C., coatings contracted due to the dominance of the hydrophobic interactions promoting cell attachment. This is in agreement with previous observations reporting that cells generally adhere to hydrophobic surfaces. (B. D. Ratner, T. Horbett, A. S. Hoffman, S. D. Hauschka, Cell adhesion to polymeric materials: implications with respect to biocompatibility, J. Biomed. Mater. Res. A. 9 (1975) 407-422). At 20° C., coatings demonstrated hydrophilic surface properties with contact angles of 33°±3° and swelled to twice their dehydrated state. The transition of the pectin coating from a collapsed state to a swollen, hydrophilic state resulted in expansion of the culture surface and subsequent weakening of cell-surface interactions.

Materials and Methods

Fabrication of Surface-Attached Thin Films of Cross-Linked Pectin Networks

Pectin obtained from citrus peel was purchased from Acros Organics (CAS 9000-69-5). Pectin powder with a degree of esterification of 67% to 70% was dissolved in deionized water (10 mg/mL) and the suspension was stirred until the pectin was completely dissolved. Thin polymer films were prepared by spin-coating pectin solutions (10 mg/mL) on substrates, and the spinning speed and duration were adjusted to obtain the desired film thickness. Briefly, the substrates were plasma cleaned and treated with a 1% solution of 3-aminopropyltriethoxysilane in acetone to enhance polymer-substrate adhesion. The substrates were heated to 110° C. to induce condensation of the silane groups to the substrate surface. A solution of 10 mg/mL pectin in water was spun-cast on a treated substrate. The spin casting was performed in a two-step processes starting at a spinning speed of 1000 rpm for 30 s followed by 30 s of spinning at 3000 RPM to maintain uniform films with thicknesses of approximately 80 nm measured by ellipsometry. Once the films were formed and dried, cross-linking was accomplished by exposing the films to solutions of CaCl₂ in ethanol with a range of concentrations of 0.0001-1M to develop different extents of cross-linking.

Swelling Characterization

The swelling profiles of the surface-attached coatings were determined using a home-built variable-angle rotating compensator ellipsometer. A He—Ne laser with a wavelength of 633 nm was utilized as the light-source. A LaSFN9 45° prism with a refractive index of n=1.845 was used as the incident medium with the solvent being used as the transmitted medium. The experimental data obtained from ellipsometry were analyzed by generating model refractive index profiles, and the ellipsometry parameters were calculated by the matrix optical formulation. The refractive index and thickness of the films were determined using an iterative procedure (least-squares minimization). The unknown thickness and optical parameters were varied to obtain the best possible fit between the experimental measurements and the theoretical ψ and A values calculated from the Fresnel equations. (Patra, L.; Messman, J. M.; Toomey, R., On the nature of volume-phase transitions in photo-cross-linked poly (cyclopropylacrylamide) and poly (N-vinylisobutyramide) coatings. Soft Matter 2013, 9 (16), 4349-4356).

ATR-FTIR Characterization

ATR-FTIR analysis was conducted to study the chemical compositions of pectin networks. The molecular interactions between pectin and Ca²⁺ ions were investigated by monitoring the main functional groups including carboxyl and carbomethoxy groups. Moreover, changes in the molecular environment during the volume-phase transition was investigated by ATR-FTIR. (R. Gnanasambandam, A. Proctor, Determination of pectin degree of esterification by diffuse reflectance Fourier transform infrared spectroscopy, Food Chem. 68(2000) 327-332; A. Synytsya, J. Čopi\acute{k} ová, P. Matějka, V. Machovič, Fourier transform Raman and infrared spectroscopy of pectins, Carbohydr. Polym. 54 (2003) 97-106). ATR-FTIR experiments of pure and cross-linked pectin were carried out using a Nicolet 8700 FTIR spectrometer equipped with a temperature controlled multi-reflection ATR ZnSe plate. FTIR measurements were performed in absorbance mode at a resolution of 4 cm-1 over a spectral range of 400-4000 cm-1. The spectral analysis was performed by a software package (OMNIC FTIR software, version 6.0, Thermo Fisher Scientific, Madison Wis.). The data represent the mean of at least triplicate experiments.

Cell Culture/Cell Release

NIH3T3 mouse embryonic fibroblast cells were purchased from the American Type Culture Collection. Dulbecco's modified Eagle's medium (DMEM), Dulbecco's phosphate buffered saline (DPBS), newborn calf serum (NCS), 0.25% trypsin EDTA, penicillin and streptomycin were obtained from Life Technologies. NIH3T3 mouse embryonic fibroblast cells were cultured in 10% NCS growth medium supplemented with 100 Uml⁻¹ of penicillin and 100 μggml⁻¹ streptomycin at 37° C. in a fully humidified atmosphere of 5% CO2 in air. Trypsinized cells were seeded onto the glass coverslips coated with cross-linked (0.1M CaCl2) pectin and glass coverslip at a density of 100 cellsmm-2 and cultured at 37° C. for 48 h. For fluorescence labeling, cultured cells were incubated with pre-warmed CellTracker™ Green CMFDA dye (Molecular probes, USA) at a concentration of 10 μM in a serum-free medium for 45 min. The dye solution was then replaced with a fresh medium and cells were incubated for an additional 30 min. The temperature of the cell culture systems was decreased by exchanging the medium with fresh medium at 20° C. and maintained at that temperature for the duration of the experiment. Cell spreading and morphology were monitored via time-lapse image acquisition on a fluorescence microscope, Eclipse Ti-U (Nikon Instruments, Japan). The cell images were analyzed with NISElements Advanced Research software Ver. 4.20 (Nikon Instruments).

Atomic Force Microscopy

Atomic force microscopy (AFM) was used to assess the surface topography and roughness of the coatings. Images were acquired with a Digital Instruments (DI-3100) scanning probe microscope in the Tapping Mode, using a Tap300A1 (Budget Sensors, Bulgaria) cantilevers with a force constant of 40 N/m. Scans of 2 μm×2 μm were recorded at a scan rate of 1 Hz.

Contact Angle Measurements

The contact angles of cross-linked coatings were measured at 20° C. and 37° C. using a KSV instrument, CAM 101 compact contact angle meter system (Kyowa Surface Chemistry Co., Ltd), and analyzed using the curve fitting software in KSV CAM Optical Contact Angle.

Conclusion

Ultra-thin coatings of cross-linked high-methoxyl pectin polysaccharide networks were fabricated by first spin-coating the polysaccharide followed by exposure to CaCl₂ in ethanol, which allowed for varying the degree of cross-linking. The responses of the thin coatings were studied as a function of temperature using ellipsometry and ATR-FTIR. The ATR-FTIR spectra comparisons of pure versus cross-linked pectins revealed that increasing CaCl₂ concentration dramatically changed the absorption band of carboxylate groups demonstrating the contribution of carboxylic acid groups in the binding of Ca⁺² ions. Moreover, the swelling of the coatings showed a pronounced de-swelling transition with increasing temperature, which is related to dehydration of the carbomethoxy groups. The coatings of cross-linked pectin networks demonstrated a tunable response with respect to the extent of cross-linking. Therefore, establishing a method to systematically alter the cross-link density is a promising alternative to conventional fabrication methods and leads to pectin hydrogel with improved properties suitable for the pharmaceutical and biotechnology industries.

### EXAMPLE 2

### Cutin Thin Films

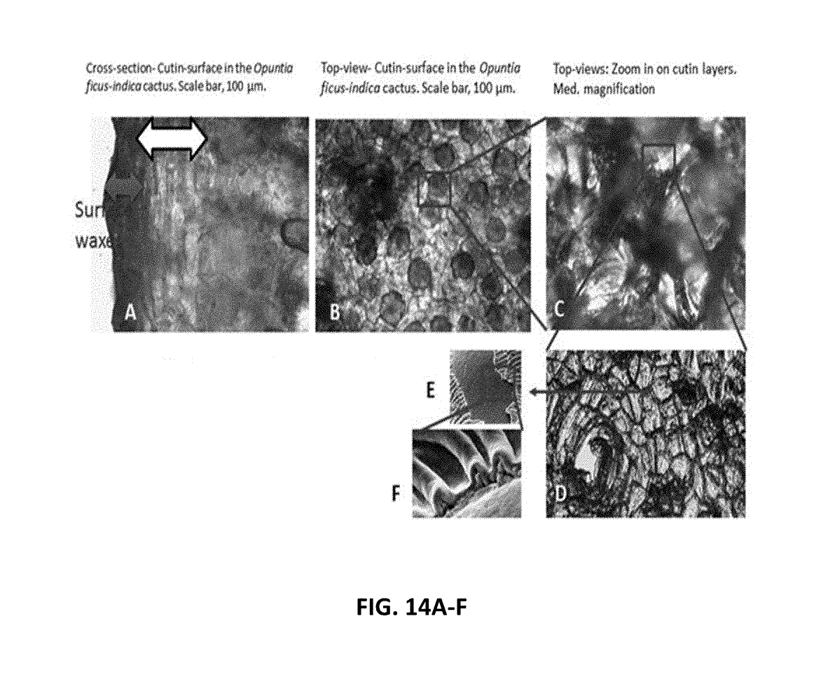

FIG. 14A-F depicts images of natural cutin. Images of natural cutin: The 3 top images (FIGS. 14A, B, C) are from the cutin layers showing reflecting and protecting properties. The lower right picture also corresponds to cutin (FIG. 14D). The small lower left images were taken at high mag. (FIGS. 14E & F). The wrinkles are surface waxes. The smooth region shows cutin nanolayers.

Natural cutin polymeric arrays are found on the outer surfaces of leaves and shoots of cacti. In natural processes, cutin, a non-living substance, results from the polymerization of fatty acids in the presence of oxygen. In particular, the composition of cutin found in the outer layer of cactus pads and shoots is of interest in 2-D layered technology development. Cactus epidermis and cuticles are capable of withstanding UV radiation damage over long periods of time (>100 years), and they tolerate relatively high and low temperatures, endure abrupt changes of temperature, withstand water damage, and reflect light. Natural cutin surfaces are extremely stable, as they do not crack nor corrode like other synthetic materials under the same extreme weather conditions. Moreover, cutin-like materials can be engineered to be amorphous or crystalline and readily impregnated with metals and metal oxides materials.

Cutin layers are highly hydrophobic and impermeable to aqueous media but susceptible to being functionalized in solution making their manufacturing flexible and inexpensive. The advanced cutin thin protective films can be applied on any surface type and texture, for example, they can be applied to wind turbines to protect them from low extreme temperatures with the synthesis and application process costing less than $100 per turbine. Coatings such as those described herein would have great interest in the energy sector since low cost technologies can be deployed in both new and existing wind farms. The films themselves are extremely light and do not increase the friction of wind turbines maintaining efficiency constant. Light reflection and thermal conductivity are minimal in cutin layers as shown in FIG. 14.

Cutin layers generate robust and biologically active substrate platforms with C—C backbones and ester bonding that can provide additional surface strength and protection. Formation of large areas of 2-D layered C—C and ester structures with distinct properties can be attained by using acid- or base catalyzed controlled ratios of cutin precursors in the presence glycerol and oxygen donor groups. These films are extremely robust and can last for a long time under extreme weather conditions with recoating being done depending on how the change in thickness evolves with time.

### Conclusion

Advanced cutin like protective films can be economically manufactured for application on various surfaces to protect the surface. These films can be made from cutin precursors in the presence of glycerol and oxygen donor groups.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall there between. Now that the invention has been described,

What is claimed is:

1. A method of manufacturing a tunable crosslinked thin film coating comprising:
obtaining a polysaccharide having a high methoxyl content;
spin-coating the polysaccharide onto a substrate to form a thin film; and
exposing the thin film polysaccharide to a concentration of calcium chloride (CaCl₂) in ethanol to produce a crosslinked thin film coating.

2. The method of claim 1, wherein the polysaccharide is a heteropolysaccharide.

3. The method of claim 2, wherein the heteropolysaccharide is pectin.

4. The method of claim 3, wherein the pectin has a degree of esterification of between about 67-70%.

5. The method of claim 1, further comprising adjusting the concentration of CaCl₂ to tune an amount of crosslinking in the crosslinked thin film coating.

6. The method of claim 1, further comprising adjusting temperature the crosslinked thin film coating is exposed to in order to tune thickness of the crosslinked thin film coating.

7. The method of claim 1, further comprising adjusting a pH of a solution the crosslinked thin film coating is exposed to in order to tune thickness of the crosslinked thin film coating.

8. A crosslinked thin film coating comprising:
a polysaccharide crosslinked with calcium chloride (CaCl₂) wherein the crosslinked thin film coating is produced by the process comprising
obtaining a polysaccharide having a high methoxyl content;
spin-coating the polysaccharide onto a substrate to form a thin film; and
exposing the thin film polysaccharide to a concentration of CaCl₂ in ethanol to produce a crosslinked thin film coating.

9. The crosslinked thin film coating of claim 8, wherein the polysaccharide is a heteropolysaccharide.

10. The crosslinked thin film coating of claim 9, wherein the heteropolysaccharide is pectin.

11. The crosslinked thin film coating of claim 10, wherein the pectin has a degree of esterification of between about 67-70%.

12. The crosslinked thin film coating of claim 8, wherein an amount of crosslinking of the crosslinked thin film coating can be tuned by changing the concentration of CaCl_{2.}

13. The crosslinked thin film coating of claim 8, wherein thickness of the crosslinked thin film coating can be tuned by changing temperature to which the crosslinked thin film coating is exposed.

14. The crosslinked thin film coating of claim 8, wherein thickness of the crosslinked thin film coating can be tuned by changing a pH of a solution to which the crosslinked thin film coating is exposed.

15. A thermo-responsive crosslinked coating for a substrate capable of use in cell culture and release applications comprising:
a polysaccharide crosslinked with calcium chloride (CaCl₂) coating wherein the crosslinked coating is produced by the process comprising obtaining a polysaccharide having a high methoxyl content;
spin-coating the polysaccharide onto a substrate to form a thin film; and
exposing the thin film polysaccharide to a concentration of CaCl₂ in ethanol to produce a crosslinked coating;
wherein at least one cell is adhered to the thermo-responsive crosslinked coating on the substrate;
wherein changing cell culture temperature changes an adherence level of the at least one cell to the thermo-responsive crosslinked coating on the substrate.

16. The coating of claim 15, wherein the polysaccharide is pectin.

17. The coating of claim 16, wherein the at least one cell is incubated at the cell culture temperature of about 37° C. to adhere the at least one cell to the thermo-responsive crosslinked coating.

18. The coating of claim 17, wherein lowering the cell culture temperature to about 20° C. detaches the at least one cell from the thermo-responsive crosslinked coating.
